(19) United States
(12) Patent Application Publication
(10) Pub. No.: US 2007/0021816 A1
(43) Pub. Date: Jan. 25, 2007

(54) Stent vascular intervention device and methods for treating aneurysms

(75) Inventor: Stephen Rudin, Williamsville, NY (US)

Correspondence Address: NIXON PEABODY LLP - PATENT GROUP, CLINTON SQUARE, P.O. BOX 31051, ROCHESTER, NY, 14603-1051, US

(73) Assignee: The Research Foundation of State University of New York, Amherst, NY (US)

(21) Appl. No.: 11/490,415

(22) Filed: Jul. 20, 2006

Related U.S. Application Data
(60) Provisional application No. 60/701,271, filed on Jul. 21, 2005.

(51) Int. Cl. A61F 2/06

(52) U.S. Cl. 623/1.4

(57) ABSTRACT

The present invention relates to a stent including a variable porosity, tubular structure having pores defined by structural surfaces. The tubular structure has a low porosity region in proximity to or at either end of the tubular structure, where the low porosity region is less porous than other regions located on the tubular structure and fully or partially obstructs passage of fluid. Any arcuate path that starts at one point within the low porosity region and goes around the perimeter of the tubular structure to stop at the same point within the low porosity region must have at least a portion that is outside of the low porosity region. Also disclosed is a method of modifying blood flow within and near an opening of an aneurysm in a blood vessel by deploying one or more stents of the present invention near an opening of the aneurysm in a blood vessel.

This application claims the benefit of U.S. Provisional Patent Application Ser. No. 60/701,271, filed Jul. 21, 2005, which is hereby incorporated by reference in its entirety.

This work was in part supported by the National Institutes of Health (Grant Nos. R01 EB002873 and R01 NS43024). The U.S. Government may have certain rights in this invention.

### FIELD OF THE INVENTION

The present invention relates to medical devices, stents in particular, and methods of treating cerebrovascular aneurysms using endovascular deployment of such stents.

### BACKGROUND OF THE INVENTION

After heart disease and cancer, stroke is the leading cause of death and adult disability in the United States. After stenoses due to plaque or thrombosis, aneurysms and their rupture is the leading cause of stroke. An intracranial aneurysm is a bulge in an artery of the brain that is prone to rupture. A ruptured intracranial aneurysm may lead to subarachnoid hemorrhage (SAH) with a high mortality rate. More than 27,000 people in America suffer from ruptured intracranial aneurysms each year (Kassell et al., “The International Cooperative Study on the Timing of Aneurysm Surgery. Part 1: Overall Management Results,” J. Neurosurg., 73:18-36 (1990)). It is generally believed that the intracranial aneurysm is initiated and developed by the hemodynamic interactions between blood flow and vessel walls. Cerebral aneurysms are most likely to be roughly round berry or saccular shaped rather than fusiform and are most likely to occur near a vessel bifurcation (Hademenos, “Saccular Aneurysm,” The Physics of Cerebrovascular Diseases, Chap. 6.4, p. 183, Springer-Verlag, New York (1998)). What is unique about aneurysms in the cerebrovasculature is that they are often formed in vessels, which have many small but important side branches or perforators. Perforators, typically about 50-250 microns in diameter, are end vessels in that they go directly to a portion of brain tissue with no co-laterals. Hence, they are the only source of blood to these regions. Should perforators be injured or disrupted, impaired brain function or death may occur.

The current treatment for neurovascular aneurysms is either invasive surgical clipping or endovascular embolization (Hademenos, “Treatment for Intracranial Aneurysms,” The Physics of Cerebrovascular Diseases, Chap. 6.8, pp. 215-223, Springer-Verlag, New York (1998); Ringer et al., “Current Techniques for Endovascular Treatment of Intracranial Aneurysms,” in Loftus et al. (eds.) Seminars in Cerebrovascular Disease and Stroke, Vol. 1(1) W.B. Saunders Company (2001)). Because invasive surgical clipping can result in substantial morbidity and mortality, catheter-based interventional procedures are becoming increasingly favored and may be the only treatment possible for some types of lesions deep within the brain. The only presently approved endovascular method is the introduction of short lengths of wire, which have thin hair-like wires sticking out the side giving them a fuzzy appearance. They are also made to bend into specified diameters when they are delivered out of the catheter tip. Thus, it is expected that these “detachable coils” will be wound around the volume of an aneurysm filling the volume of the aneurysm without herniating out into the main blood vessel. If enough of these coils are placed in the aneurysm to disrupt the vortex-like blood flow, it is expected that the blood remaining in the aneurysm adjacent to the coils will thrombose and that a layer of endothelial cells at the neck or entrance to the aneurysm will begin the process of the formation of a new wall to the vessel (Langille, “Blood Flow-Induced Remodeling of the Artery Wall,” in Bevan (eds.) Flow-Dependent Regulation of Vascular Function, Ch. 13, pp. 277-299, Oxford University Press, New York, N.Y. (1995)). The aneurysm, with the coil mass within, is thus sealed off and the main vessel is, in the ideal case, fully recanalized or remodeled to allow normal laminar-like blood flow to resume.

In practice, there are a number of problems with this scenario. The coils may not fully fill the aneurysm volume, since the ones deployed first may interfere with the deployment of the later ones. It may take many coils of different length and diameter to come near to filling the aneurysm volume. A coil may herniate into the main vessel and cause thrombi to form. If these thrombi stay in the main vessel and travel further into the brain, an ischemic stroke may result. Also, one of the coils may inadvertently perforate a weak section of the aneurysm wall resulting in catastrophic hemorrhage. Positioning the final coils may shift the first coils around to undesired positions, either preventing further coiling to completion or possibly causing herniation or perforation. Compaction may commonly occur in time having the effect of incomplete neck filling. The disruption of aneurysmal blood flow may be inadequate and the aneurysm or a new one may regenerate in the same location. Treatment of large and giant aneurysms with coils has been problematic. Additionally, if the aneurysm has a wide neck or is fusiform (bulging on all sides with no clearly defined neck), it may not be possible to introduce coils that will remain within, thus precluding this type of treatment. Finally, there is a growing concern about long-term incomplete endothelialization across the neck resulting from coiling (Bavinzski et al., “Gross and Microscopic Histopathological Findings in Aneurysms of the Human Brain Treated With Guglielmi Detachable Coils,” J. Neurosurg., 91:284-293 (1999); Reul et al., “Long-Term Angiographic and Histopathologic Findings in Experimental Aneurysms of the Carotid Bifurcation Embolized With Platinum and Tungsten Coils,” Am. J. Neuroradiol., 18:35-42 (1997); Kallmes et al., “Histologic Evaluation of Platinum Coil Embolization in an Aneurysm Model in Rabbits,” Radiology, 213:217-222 (1999)).

One approach that is being pursued by Micro Therapeutics, Inc. (Irvine, Calif.) is the use of a liquid polymer material instead of coils. Because the liquid polymer is so viscous, a special high-pressure micro-catheter must be used and placed in the aneurysm, while the orifice of the aneurysm, as well as the main vessel, is blocked by a balloon. The polymer is then introduced into the aneurysm and prevented from escaping into the main vessel by the inflated balloon. The aneurysm is filled in stages every few minutes. Only a few tenths of a milliliter flows into the aneurysm, before the balloon must be deflated to allow blood to resume flowing into the main vessel. Before the next stage, there is a pause while the polymer solidifies after which new liquid polymer is introduced until the aneurysm is finally filled. The balloon does not form a perfect seal to allow displaced blood to leave, but unfortunately at the end of the procedure when the aneurysm is filled, often the polymer flows out over the balloon forming flaps in the main vessel. The potential consequences of this are not known and this procedure is not yet FDA approved. One advantage of the method is that the balloon enables treatment of wide necked aneurysms not possible with coils. The disadvantages aside from the flap formation is the need to repeatedly stop blood flow in the main vessel, the lengthy duration of time needed for the procedure, and the possibility of technical complications such as solidification of the polymer and clogging of the special catheter.

During the attempt to treat wide-necked aneurysms with coils, researchers have tried coils in combination with stents (Szikora et al, “Combined Use of Stents and Coils to Treat Experimental Wide-Necked Carotid Aneurysms: Preliminary Results,” Am. J. Neuroradiol., 15:1091-1102 (1994); Lanzino et al., “Efficacy and Current Limitations of Intravascular Stents for Intracranial Internal Carotid, Vertebral, and Basilar Artery Aneurysms,” J. Neurosurg., 91:538-546 (1999)). Stents are cylindrical scaffolds usually made of stainless steel or nitinol, which are generally used for the treatment of stenoses or vessel narrowing due to atherosclerosis. For application to the endovascular treatment of aneurysms, the stent's function is not one of holding the vessel open but of preventing the coils inserted in an aneurysm from herniating out into the main vessel. The struts of the stent are placed over the orifice of the aneurysm to act as a barrier. Researchers have demonstrated that merely by the deployment of a stent across the ostium of an aneurysm, the characteristic vortex blood flow would be reduced (Lieber et al., “Alteration of Hemodynamics in Aneurysm Models by Stenting: Influence of Stent Porosity,” Annals Biomed. Eng., 25:460-469 (1997); Aenis et al., “Modeling of Flow in a Straight Stented and Non-Stented Side Wall Aneurysm Model,” J. Biomech. Eng., 119:206-212 (1997); Livescu et al., “Intra-Aneurysmal Vorticity Reduction Subsequent to Stenting,” Annals Biomed. Eng., Vol. 28, Supp. 1:S-61, BMES 2000 Annual Fall Meeting, Seattle, Wash. (2000); Livescu et al., “Influence of Stent Design on Intra-Aneurysmal Flow—A PIV Study,” in Conway (ed.) 2000 Advances in Bioengineering, BED, Vol. 48, ASME Publication: 3-4, International Mechanical Engineering Conference & Exposition 2000, Orlando, Fla. (2000); Nichita et al., “Numerical Simulation of Flow in a Stented and Non-Stented Side Wall Aneurysm Model Using the Immersed Boundary Technique,” Annual Meeting of the Society for Mathematical Biology (SMB 2000), Salt Lake City, Utah (2000); Nichita et al., “Numerical Simulation of Flow in a Stented and Non-Stented Cerebral Arterial Segment with a Side Wall Aneurysm Using the Immersed Boundary Technique,” Annals Biomed. Eng., Vol. 28, Supp. 1:S-61, BMES 2000 Annual Fall Meeting, Seattle, Wash. (2000)). It was found that the porosity, or open area compared to total outside area of the cylindrical stent, determined how much disruption of the vortex occurred. In one clinical case, where only a stent was deployed with no coils, it was found that the aneurysm actually self-thrombosed (Hopkins et al., “Treating Complex Nervous System Vascular Disorders Through a “Needle Stick”: Origins, Evolution, and Future of Neuroendovascular Therapy,” Neurosurgery, 48:463-475 (2001)).

Results of aneurysm stenting have been inconsistent. Geremia et al. deployed self-expanding, cobalt-alloy stents in sidewall aneurysms and fusiform aneurysms of canine models (Geremia et al., “Embolization of Experimentally Created Aneurysms With Intravascular Stent Devices,” Am. J. Neuroradiol., 15:1223-1231 (1994)). Near-complete ablations were observed eight weeks after stent placement while the stented carotid arteries remained widely patent. They concluded that a woven wire stent can alter the aneurysmal blood flow patterns, and promote the formation of thrombus and fibrosis within the residual aneurysmal lumen. Vanninen et al. reported that complete thrombosis was induced by stent placement in three saccular aneurysms of patients, without additional packing of the aneurysm with coil (Vanninen et al., “Broad-Based Intracranial Aneurysms: Thrombosis Induced by Stent Placement,” Am. J. Neuroradiol., 24:263-266 (2003)). Recently, Krings et al. treated elastase induced rabbit aneurysms with covered stents as well as porous stents (Krings et al., “Treatment of Experimentally Induced Aneurysms with Stents,” Neurosurgery, 56:1347-1359 (2004)). Covered stents induced complete obliterations of the most aneurysms, but they found the parent vessel occlusion for one in the three-month follow-up group. Porous stents led to the aneurysm occlusion in two of five aneurysms in the one-month follow-up group, and four of five aneurysms in the three-month follow-up group. Lanzino et al. originally treated four patients' aneurysms with porous stents solely (Lanzino et al., “Efficacy and Current Limitations of Intravascular Stents for Intracranial Internal Carotid, Vertebral, and Basilar Artery Aneurysms,” J. Neurosurg., 91:538-546 (1999)). No evidence of aneurysm thrombosis was observed either immediately after the procedure or on follow-up angiographic studies.

It has become somewhat common practice now to deploy stents in combination with detachable coils. In many such cases, the stent is first deployed and then a microcatheter to deliver the coils is inserted through the openings between the struts of the stent. Nevertheless, many of the potential disadvantages of using coils, such as risk of perforation, long duration of procedure, incomplete filling of the volume, and regrowth of the aneurysm (Hayakawa et al., “Natural History of the Neck Remnant of a Cerebral Aneurysm Treated With the Guglielmi Detachable Coil System,” J. Neurosurg., 93:561-568 (2000)) remain; in addition, there is the new risk to perforator vessels whose orifice may be in close proximity to the aneurysm and hence covered by stent struts. Most recently, there has been a case where adverse effects possibly attributed to blood flow pattern changes occurred. However, detailed flow patterns and consequential wall stress fields, even though generally believed to be crucial to the occurrence, progression, and recurrence after therapy of neurovascular aneurysms (Imbesi et al., “Analysis of Slipstream Flow in a Wide-Necked Basilar Artery Aneurysm: Evaluation of Potential Treatment Regimens, Am. J. Neuroradiol., 22:721-724 (2001); Sorteberg et al., “Effect of Guglielmi Detachable Coils on Intraaneurysmal Flow: Experimental Study in Canines,” Am. J Neuroradiol., 23:288-294 (2002)) are mostly unexplored.

Many aneurysms occur on curved vessels at bifurcation or trifurcation points in the vessel tree. In addition, wide necked bifurcation aneurysms are currently very difficult to treat. Such aneurysms may not be optimally treatable by any of the methods described above, because of the complication rate and the risk of invasive surgical procedures, the difficulty in placing the stent in front of the aneurysm neck, or because the neck of the aneurysm is too wide or the aneurysm is too large or delicate.

While stenting may provide a new, less invasive therapeutic option for cerebral aneurysms, a conventional porous stent may be insufficient in modifying the blood flow for clinical aneurysms. That is because the original primary purpose of stents is to support the wall of the diseased vessel rather than modify blood flow; thus, all commercially available stents are uniform and circularly symmetric. Clearly this is not an ideal design for treatment of neurovascular aneurysms which are inherently non-radially symmetric, since they are either bulges in the side of a vessel wall or bulges at a vessel bifurcation or fusiform but asymmetric in shape. A uniformly covered stent would be fatal since it would cover perforators as well as the aneurysm orifice.

The present invention is directed to overcoming the above-noted deficiencies in the art.

### SUMMARY OF THE INVENTION

The present invention relates to a stent including a variable porosity, tubular structure having pores defined by structural surfaces. The tubular structure has a low porosity region in proximity to or at either end of the tubular structure, where the low porosity region is less porous than other regions located on the tubular structure and fully or partially obstructs passage of fluid. The low porosity region is larger than the structural surfaces between adjacent pores. Any arcuate path that starts at one point within the low porosity region and goes around the perimeter of the tubular structure to stop at the same point within the low porosity region must have at least a portion that is outside of the low porosity region.

Another aspect of the present invention relates to a method of modifying blood flow within and near an opening of an aneurysm in a blood vessel. The method involves deploying one or more of the above stents according to the present invention near an opening of an aneurysm in a blood vessel, so that the low porosity region of the stent causes modification of blood flow within and near the opening of the aneurysm.

The stent of the present invention is advantageous in that it enables somewhat straightforward treatment of difficult to treat aneurysms that are inherently non-uniform and non-symmetric in nature. For difficult cases of aneurysms, such as bifurcation or trifurcation aneurysms or where the aneurysm may be wide and not suitable to being treated by any of the existing methods, the stent of the present invention could be used to retard or eliminate flow into the aneurysm without risking filling the aneurysm and causing possible rupture. Even the treatment of basilar tip aneurysms with narrow necks by multiple coil insertion could be shortened in duration by the simple accurate deployment of the stent of the present invention. In the case of a wide neck basilar tip or any other bifurcation aneurysm, it is not possible to keep a coil mass in place nor is it possible to deploy a single stent in front of the aneurysm opening. Especially, for a basilar artery tip where the basilar artery leads into the two posterior cerebral arteries at almost a 90 degree angle, there is no way to deploy a stent to cross between the two posterior communicating cerebral arteries such that the body of the stent lies in front of the aneurysm opening. If two of the asymmetric stents according to the present invention are used, they can be deployed into the two posterior communicating cerebral arteries so that the low porosity patches at the proximal end of the stents meet to retard blood flow into the aneurysm while the stents would be anchored further up along each of the posterior communicating cerebral arteries. Similarly for other aneurysms at other vessel bifurcations, one or more asymmetric stents according to the present invention could be deployed relatively easily yet with great effect on aneurysmal blood flow.

### BRIEF DESCRIPTION OF THE DRAWINGS

FIGS. 1A-B show two different views of an exemplary stent of the present invention having a low porosity region.

FIG. 2 shows another exemplary stent of the present invention that was created by attaching a low porosity stainless steel cloth (500 wires per inch; cloth porosity (open area compared to total outside area of the stent) 25%; thickness 50 μm) onto a Penta coronary stent (Guidant Corp., Temecula, Calif.) by laser micro welding and then attaching four platinum markers (indicated by arrows in the figure and inset; diameters ranging from 100 to 150 μm) to indicate the position of the asymmetric low porosity region. The stent was crimped onto a balloon tipped catheter, where the diameter of the stent was 1.5 mm when crimped onto the balloon. The stent on the catheter was inserted into a 6 Fr introducer placed in the femoral artery and used for in vivo experiments.

FIGS. 3A-B are schematic diagrams of two different views of a bifurcation aneurysm where two stents of the present invention are shown deployed.

FIG. 4 illustrates how two stents of the present invention can be deployed in a bifurcation aneurysm where the aneurysm is located more toward the smaller branch vessel.

FIG. 5 illustrates how two stents of the present invention can be deployed in a bifurcation aneurysm where the aneurysm is located more toward the larger main vessel.

FIG. 6 illustrates how two stents of the present invention can be deployed in a bifurcation aneurysm where the aneurysm is located at the split of a main vessel into two branch vessels.

FIG. 7 shows two images of ideal aneurysm models where the aneurysm orifice is partially covered by the low porosity region of the stent of the present invention (see top two images), as well as the corresponding results of computational fluid dynamics (CFD) calculations (see bottom two images) on the two models whose images appear above each.

FIG. 8 shows three images of patient-specific aneurysms derived from computed tomography (CT) scan data where the aneurysm is untreated, the proximal neck blocked, and the distal neck blocked by the low porosity region of the stent of the present invention (see top left, middle, and right images, respectively), as well as the corresponding results of CFD calculations (see bottom three images) on the three models whose images appear above each.

FIG. 9 shows the geometries of an anterior cerebral artery aneurysm of a specific patient (left) and an asymmetric stent with a patch designed to block the inflow jet at the proximal neck of the aneurysm (right).

FIG. 10 depicts the velocity wave of the pulsatile flow. The solid line indicates the contrast agent injection.

FIG. 11 depicts a specially designed asymmetric stent with a low porosity patch for treatment of the patient-specific aneurysm.

FIG. 12 shows the particle paths in the steady state flow simulations in the untreated and the stented aneurysm models.

FIG. 13 illustrates the instantaneous aneurysm wall shear stress distributions for the untreated and the stented aneurysm models.

FIGS. 14A-D depict the visualization of aneurysmal inflow using digital subtraction angiography (DSA) and CFD virtual angiographic images: untreated-DSA (FIG. 14A); stented-DSA (FIG. 14B); untreated-CFD (FIG. 14C); stented-CFD (FIG. 14D).

FIGS. 15A-D depict the visualization of the instantaneous images of the contrast medium in the aneurysm at a later time than that depicted in FIGS. 14A-D (at time=0.5 sec, systole): untreated-DSA (FIG. 15A); stented-DSA (FIG. 15B); untreated-CFD (FIG. 15C); stented-CFD (FIG. 15D).

FIG. 16 shows the variation of the average concentration of the contrast medium in the aneurysm. DSA data was normalized for a comparison: (A) untreated-DSA; (B) stented-DSA; (C) untreated-CFD; (D) stented-CFD.

FIG. 17 illustrates the velocity vectors on a plane across the middle of the patient-specific aneurysm in an untreated and stented case.

### DETAILED DESCRIPTION OF THE INVENTION

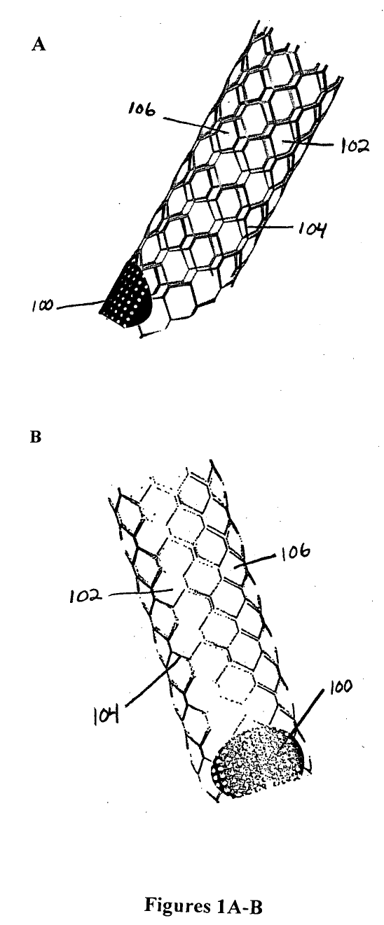

The present invention relates to a stent including a variable porosity, tubular structure having pores defined by structural surfaces. FIGS. 1A-B show two different views of an exemplary stent of the present invention.

As shown in FIGS. 1A-B, the tubular structure of the stent of the present invention has low porosity region 100 in proximity to or at either end of the tubular structure, where low porosity region 100 is less porous than other regions 102 located on the tubular structure and fully or partially obstructs passage of fluid. Low porosity region 100 is larger than structural surfaces 104 between adjacent pores 106. Any arcuate path that starts at one point within the low porosity region and goes around the perimeter of the tubular structure to stop at the same point within the low porosity region must have at least a portion that is outside of the low porosity region. The phrase “arcuate path” used herein means a path that is curved including, but not limited to, circular-shaped and elliptical-shaped paths on the surface of the tubular structure.

The present invention provides a stent with an asymmetric low porosity region capable of modifying blood flow so as to change the hemodynamic conditions that result in aneurysms or any other flow-related pathology. Thus, the main body of the stent of the present invention is used to secure the position of the stent in the vasculature so that the low porosity region in proximity to or at either end of the stent can be held in place to cause the flow modification. Accordingly, the shape of the cut at the end of the stent is adapted to the morphology of the vessel structure. For example, an end of the stent can be obliquely cut using a flat plane, with an additional cut perpendicular to the stent axis so as to cut off the pointed tip and form a chamfered shape at the end of the stent where the low porosity region may reside. In actuality, however, the end of the stent should conform to the requirements of the specific patient morphology. Thus, it might be advisable to cut the end of the stent using a curved plane. This plane could begin as a cut perpendicular to the stent axis but conclude at an oblique angle with a continuously curved cutting surface in between. This embodiment could also have a chamfered-like end or the tip could be further rounded rather than be a straight line chamfer. Thus, in another embodiment of the present invention, the end of the tubular structure of the stent of the present invention has a shape optimal for use inside a blood vessel and/or with another stent.

In another embodiment of the present invention, the tubular structure of the stent of the present invention has a generally cylindrical shape, where all cross sectional areas of the tubular structure that are perpendicular to the longitudinal axis of the tubular structure have circular shapes with identical diameters. Alternatively, the stent of the present invention can have diameters that change from one end to the other so as to better fit the changing shape of the actual vessel being treated, since for example in some blood vessels the parent vessel starts out with a larger diameter proximal to the aneurysm and is reduced in diameter distal to the aneurysm. Thus, in another embodiment of the present invention, all cross sectional areas that are perpendicular to the longitudinal axis of the tubular structure of the stent have circular shapes with variable diameters. Specifically, the tubular structure can have a frusto-conical shape. In other embodiments of the present invention, cross sectional areas of the tubular structure of the stent that are perpendicular to the longitudinal axis of the tubular structure have variable shapes, such as elliptical or oval shapes and any irregular shape.

In another embodiment of the present invention, the low porosity region can be formed by a polymer membrane patch attached to the tubular structure of the stent of the present invention, as depicted in FIGS. 1A-B. The polymer membrane patch can be made of any type of biocompatible membrane material, such as polyurethane and polytetrafluoroethylene. For example, polyurethane can be applied as a liquid to an existing symmetric stent from one of the commercial manufacturers where it dries into a film or membrane for the asymmetric low porosity patch region. The polyurethane liquid can be applied so that the patch boundaries coincide with the struts of the stent. The combination of a self-expanding stent with the above-described polymer membrane patch may provide the most practical application of the present invention to human clinical treatments, because currently available balloon expandable stents tend to be too stiff or inflexible mechanically for consistent application to deep cerebral vessels. Although FIGS. 1A-B depict a specific embodiment of the stent of the present invention where the low porosity patch has a uniform distribution of holes, other embodiments of the present invention are also possible where the porosity of the low porosity patch is variable, e.g., lower in the center and the end of the patch and higher toward the other regions (i.e., higher porosity region) of the stent, so as to protect any perforator sidewall vessels that might be nearby and covered by the middle and distal end of the stent.

In another embodiment of the present invention, the tubular structure of the stent of the present invention can be a cylindrical sheet with pores of variable size or shape, as depicted in FIG. 1A of U.S. Patent Application Publication No. US 2003/0109917 to Rudin et al., which is hereby incorporated by reference in its entirety. The low porosity region can have a single pore size while all other parts of the tubular structure have another larger pore size.

Alternatively, the low porosity region of the stent of the present invention can have a plurality of pore sizes with the size of the pores increasing as the low porosity region transitions to other regions of the stent, as depicted in FIG. 1B of U.S. Patent Application Publication No. US 2003/0109917 to Rudin et al., which is hereby incorporated by reference in its entirety.

In other embodiments of the present invention, the tubular structure of the stent of the present invention can be formed from a plurality of strut elements which are thicker, wider, and/or denser in the low porosity region, as shown and described in FIGS. 1C-E and paragraphs [0021] to [0024] of U.S. Patent Application Publication No. US 2003/0109917 to Rudin et al., which is hereby incorporated by reference in its entirety. The strut elements can be made of stainless steel. In another embodiment of the present invention, the tubular structure is made of a mesh material.

In yet another embodiment of the present invention, the low porosity region of the stent is formed by flap-like structures in the pores which could be deployed or changed in the field to obstruct fluid flow, as depicted in FIG. 1F of U.S. Patent Application Publication No. US 2003/0109917 to Rudin et al., which is hereby incorporated by reference in its entirety.

The stent of the present invention can be balloon expandable so that it can be deployed using a balloon catheter. Alternatively, the stent of the present invention can be self-expandable where the stent is made of a superelastic or shape memory material and can be deployed by self-expansion. Superelastic or shape memory materials can be annealed into a first shape, heated, thereby setting the material structure, cooled, and deformed into a second shape. The material returns to the first, remembered shape at a phase transition temperature specific to the material composition. Superelastic or shape memory materials include, for example, nickel-titanium alloy, which is available under the name of nitinol.

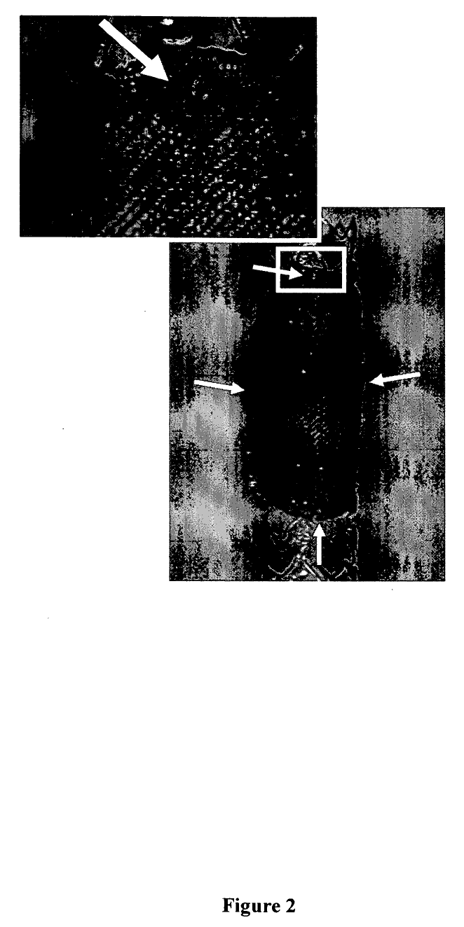

In another embodiment of the present invention, the stent of the present invention can be marked with, or at least partially made of, a radioopaque material imageable by high resolution radiographic imaging in order to aid in correctly deploying the stent. Suitable radioopaque material includes platinum, gold, tantalum, and iodine impregnated material. FIG. 2 shows a stent of the present invention, where four platinum markers (indicated by arrows in figure and inset) are used to mark the position of the asymmetric low porosity patch on the stent.

Another aspect of the present invention relates to a method of modifying blood flow within and near an opening of an aneurysm in a blood vessel. The method involves deploying one or more stents according to the present invention near an opening of an aneurysm in a blood vessel, so that the low porosity region of the stent causes modification of blood flow within and near the opening of the aneurysm. The aneurysm can be located in proximity to a vessel junction where one or more blood vessels split or merge into one or more blood vessels, such as a vessel bifurcation or trifurcation. With regard to the use of the stent of the present invention for trifurcation or other aneurysms where there are any number of vessels leading in and out of the aneurysm, the purpose of the stent of the present invention is to modify flow either going in or coming out of the aneurysm. For example, if one thinks of the bifurcation aneurysm like the common basilar artery tip aneurysm but consider an additional vessel coming out of the plane of the standard two daughter posterior communicating cerebral arteries yet with the aneurysm still at the tip, then in addition to two asymmetric stents with proximal low porosity regions in the posterior communicating cerebral arteries, one could put an additional such stent in the third vessel but with the orientation of the low porosity region approximately perpendicular to those of the two posterior communicating cerebral arteries stents. In this way, blood flow into the aneurysm could be further modified. Such trifurcation aneurysms or aneurysms with more than three or four vessels can be complex in shape and may require computer fluid dynamic calculations with virtual stents to determine what beneficial flow modification would be advisable.

The stent of the present invention can be deployed so that the low porosity region at one end of the tubular structure is proximal to the opening of the aneurysm while the other end of the tubular structure is distal to the opening of the aneurysm. Alternatively, the stent of the present invention can be deployed so that the low porosity region at one end of the tubular structure is distal to the opening of the aneurysm while the other end of the tubular structure is proximal to the opening of the aneurysm.

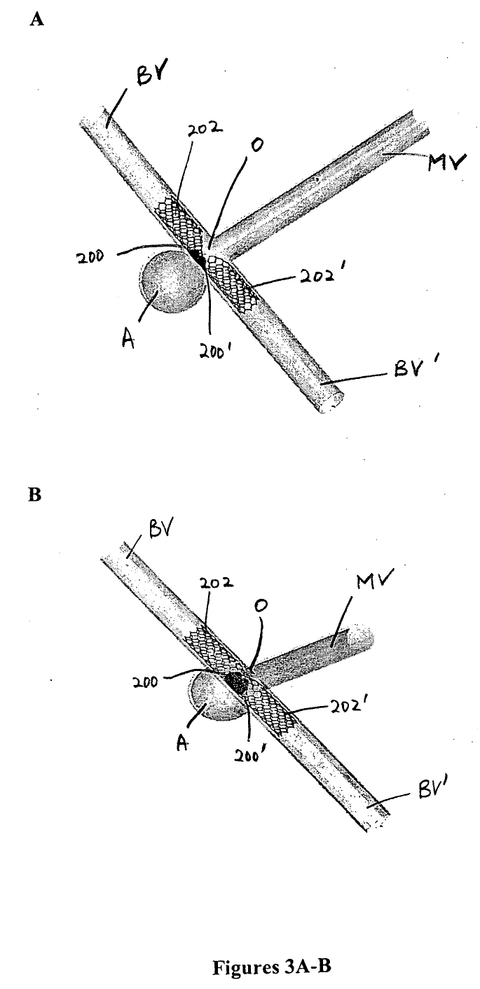

FIGS. 3A-B illustrate how two stents of the present invention can be deployed in a blood vessel near a bifurcation aneurysm. In FIGS. 3A-B, main vessel MV bifurcates at 90 degrees into two branch vessels BV, BV′, which are parallel to one another. At the tip of main vessel MV is aneurysm A. This geometry somewhat simulates a basilar artery aneurysm; however, in an actual basilar artery aneurysm, the vessels are rarely at an angle of exactly 90 degrees. Inside branch vessels BV, BV′ have been placed stents 202, 202′ where the ends of the stents proximal to aneurysm A is cut at an angle to indicate a wedge shaped point. Since the approach for any catheter to deliver stents must be through the main vessel, it would not be possible to place a single stent across the aneurysm extending along the two branch vessels. Thus, the catheter must originate in the main vessel. Each stent 202, 202′ is, therefore, deployed separately and positioned so that low porosity patches 200, 200′ at the ends of the stents proximal to aneurysm A are both facing opening O to aneurysm A, thereby acting together to severely restrict flow into aneurysm A. Both stents 202, 202′ can have one of their ends, i.e., the end proximal to the aneurysm, cut into a chamfer and then the sharp end cut again to form a somewhat smooth edge (see e.g., FIGS. 1A-B) which would be able to snuggly meet the corresponding end of the other stent.

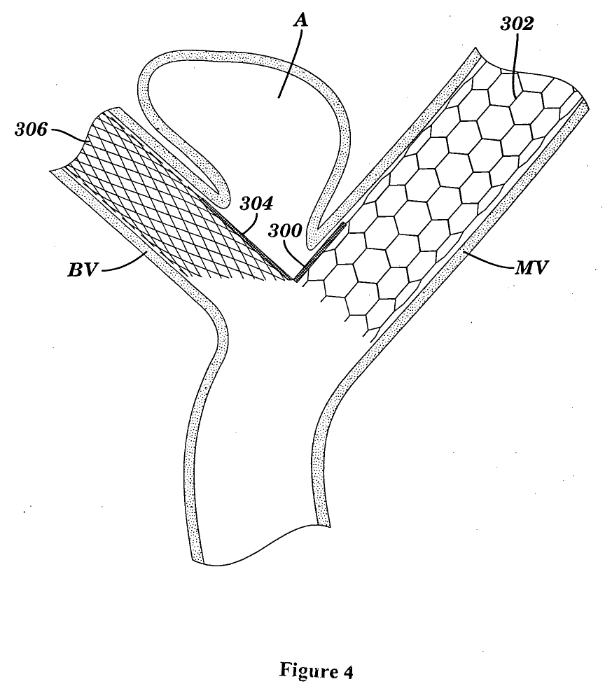

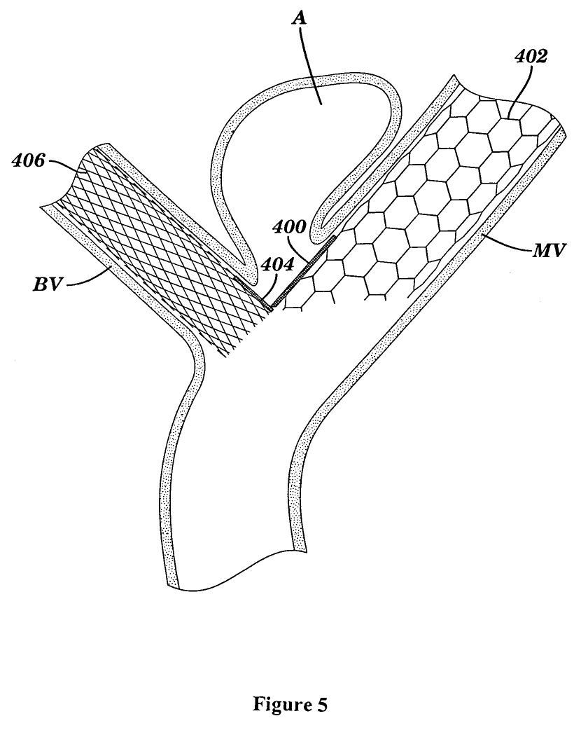

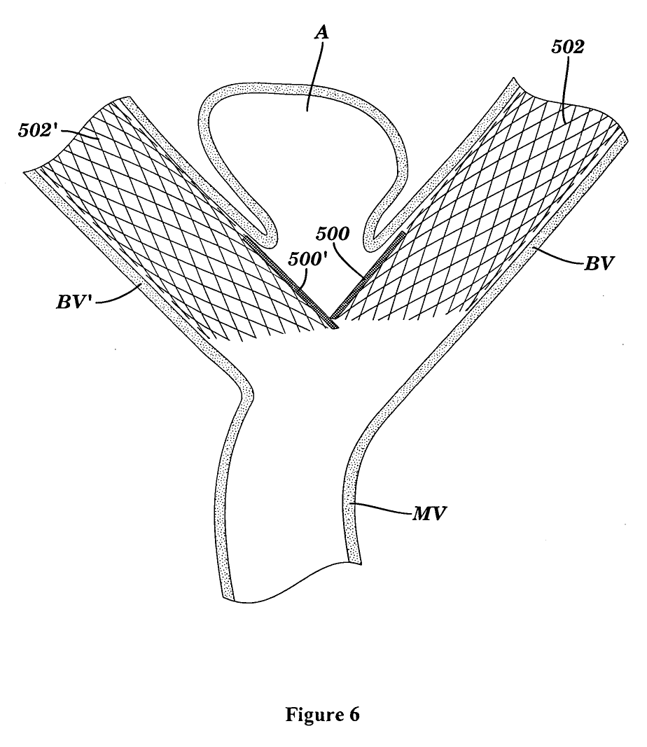

FIGS. 4, 5, and 6 show different examples of bifurcation aneurysms where stents of the present invention can be used. Specifically, FIG. 4 depicts a bifurcation aneurysm where there is large and somewhat curved main vessel MV and smaller branch vessel BV. Aneurysm A is located more toward smaller branch vessel BV. As shown in FIG. 4, two stents 302, 306 must have different diameters to fit respective vessels MV, BV and there is little overlap between two stents 302, 306. Low porosity regions 300, 304 are at the ends of the stents proximal to aneurysm A which may or may not be chamfered. The two stent structures may or may not have different porosities for the higher porosity regions of the stents. FIG. 5 depicts a bifurcation aneurysm where there is a large main vessel MV and smaller branch vessel BV. Aneurysm A is located more toward larger main vessel MV, in contrast to the aneurysm depicted in FIG. 4. As shown in FIG. 5, two stents 402, 406 must have different diameters to fit respective vessels MV, BV. Low porosity regions 400, 404 are at the ends of the stents proximal to aneurysm A and overlap. The two stent structures may or may not have different porosities for the higher porosity regions of the stents. FIG. 6 specifically depicts a roughly symmetric bifurcation with branch vessels of about the same diameter. Aneurysm A is located at the point two branch vessels BV, BV′ split from main vessel MV. As shown in FIG. 6, low porosity regions 500, 500′ are at the ends of the stents proximal to aneurysm A, which are chamfered, and overlap, although they do not have to completely overlap as long as the blood flow is sufficiently modified to reduce the growth of the aneurysm.

For each vessel bifurcation geometry, different stents of the present invention with a different shaped low porosity patch and stent chamfer angle and shape could be used that would be optimal for reducing flow into the aneurysm. Thus, this would be a patient specific treatment based upon the deployed asymmetric stents of the present invention. Additionally, if there were additional vessels at the junction such as three for a trifurcation, then there could be an appropriately designed asymmetric stent of the present invention inserted into each vessel with the end proximal to the aneurysm contributing to the restriction of blood flow into the aneurysm opening.

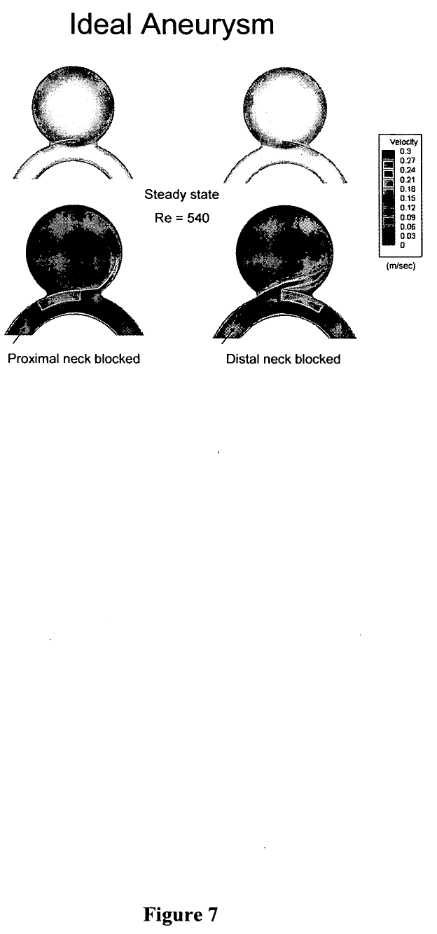

FIG. 7 shows four images of an idealized spherical aneurysm on a curved or bent vessel. In the top two images, the location of the low porosity patch is indicated with respect to the aneurysm orifice. The stent of the present invention which supports this low porosity region is itself of high porosity and is assumed not to affect the CFD calculations. The low porosity region as depicted in the upper left image could be on the distal end of a stent which is deployed in the proximal (left in the image) vessel segment, whereas the low porosity region as depicted in the upper right image could be on the proximal end of a stent which is deployed in the distal (right in the image) vessel segment. The lower two images indicate the results of the CFD calculation and how the flow into the aneurysm is modified by the two stent deployments. In the image on the lower right, the flow appears to be modified so as to protect the distal neck of the aneurysm.

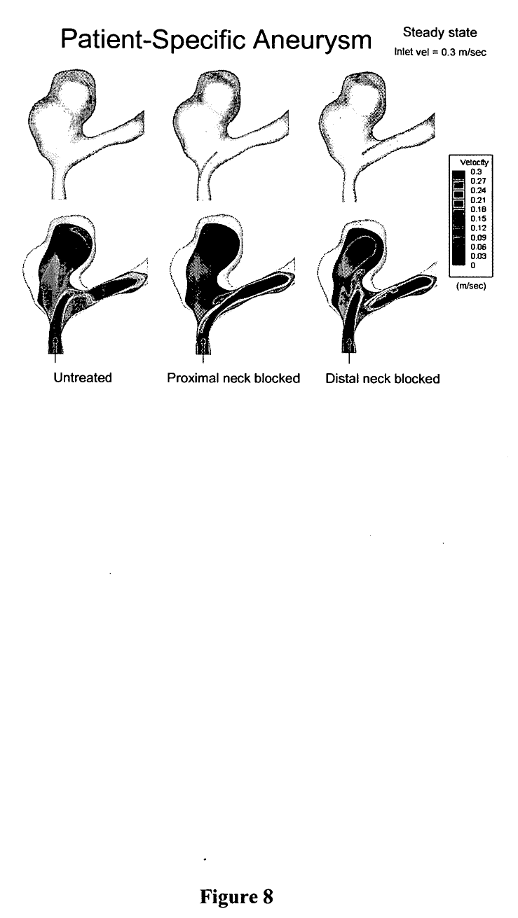

FIG. 8 shows six images of an actual human aneurysm derived from CT scan data. The upper three images indicate the location of the deployment of the low porosity region of the stent of the present invention where again the stent structure itself is not indicated because it is assumed not to have a significant effect on flow. In the first image there is no stent, in the second image the low porosity patch of the stent is proximal, and in the third image the low porosity patch of the stent is placed distally to the center of the neck of the aneurysm. The three images on the bottom of FIG. 8 show the results of the calculation for the conditions described by the three images above them, i.e., for the image on the bottom left, there is no low porosity patch, for the image on the bottom middle, the low porosity patch blocks the proximal portion of the neck, and for the image on the bottom right, the low porosity patch blocks the distal portion of the neck. It is notable how the flow is drastically modified by the proximal positioning (see image on the bottom middle) so that the jet originally impinging into the aneurysm (see image on the bottom left) is obliterated, whereas the distal positioning appears to move the jet further up into the aneurysm. Using a stent with a proximal patch that has an outcome on flow modification, such as the one indicated in the image on the bottom middle, can provide positive therapeutic effects in reducing or eliminating future aneurysm growth or rupture.

Balloon expansion and self-expansion are the most common methods of deploying stents. In one embodiment of the present invention, the stent of the present invention is deployed by self-expansion of the stent. Thus, a stent made of a superelastic or shape memory material can be used, where the stent is compressed to fit within a microcatheter, delivered to the aneurysm, and pushed from the microcatheter end. Subsequently, the stent regains its uncompressed shape, where the low porosity region of the stent is positioned near the opening of an aneurysm so as to modify blood flow within and near the opening of the aneurysm.

Part of the difficulty in present applications of stents to the cerebral vasculature is the difficulty in navigating a somewhat rigid undeployed stent through tortuous vasculature to the lesion. Part of the reason for the rigidity in stents is the requirement for treatment of stenoses that the stent maintain sufficient hoop strength to keep the vessel in question open. For application to aneurysms, however, this requirement for rigidity can be relaxed because the sole function of the stent is to only be strong enough away from the aneurysm orifice to keep the low porosity region or the patch-like region of the stent in position near or over the aneurysm orifice so as to modify the flow of blood into the aneurysm, as in the present invention.

In order to correctly deploy the stent of the present invention near the opening of an aneurysm, one would need a way to visualize the asymmetric part of the stent (i.e., the low porosity region). Thus, the stent of the present invention will have to be positioned accurately both in the direction of the catheter axis and also in rotational angle, so as to position the low porosity region of the stent near the aneurysm orifice. Therefore, another embodiment of the present invention relates to using high resolution radiographic imaging to guide the deployment of the stent of the present invention. U.S. Pat. No. 6,285,739 to Rudin et al., which is hereby incorporated by reference in its entirety, discloses high resolution micro-angiographic detectors for viewing a limited region of interest near the interventional site, usually at the catheter tip, which can be used to provide the necessary guidance for accurate rotational orientation of the stent in the blood vessel. In addition, improved methods of placing radioopaque markers on the stent that can easily be used for alignment of the stents during radiological guidance have been developed.

### EXAMPLES

The following examples are provided to illustrate embodiments of the present invention but are by no means intended to limit its scope.

### Example 1

### Evaluation of an Asymmetric Stent Patch Design

Aneurysm hemodynamics is known to be significantly affected by the arterial and the aneurysmal wall boundaries which vary from patient to patient (Rhee et al., “Changes of Flow Characteristics by Stenting in Aneurysm Models: Influence of Aneurysm Geometry and Stent Porosity,” Ann. Biomed. Eng., 30:894-904 (2002), which is hereby incorporated by reference in its entirety). Therefore, it is important to consider the specific geometrical characteristics of an artery and an aneurysm to make hemodynamically favorable modifications using placement of a stent.

An asymmetric stent patch was designed for an anterior cerebral artery aneurysm of a specific patient, where the patch porosity varied across the neck. The local porosity of the patch at the proximal neck was designed to block the strong inflow jet in the patient-specific aneurysm. The purpose of the study was to evaluate the hemodynamic effects of the patient-specific asymmetric stent patch using computational fluid dynamics (CFD) as well as digital subtraction angiography (DSA).

A cerebral aneurysm geometry of a patient was reconstructed from computed tomographic angiography (CTA) images of the patient's right anterior communicating artery (ACA). The specific hemodynamic features of this geometry were investigated using CFD models under both steady-state and pulsatile flow boundary conditions. With these results, a patient-specific asymmetric stent patch was designed to minimize the aneurysmal flow activity to enable conditions that could induce thrombosis in the aneurysm. The porosity of the patch varied both longitudinally and axially. The patch was deformed by commercial CAD software to fit into the lumen, then virtually placed across the aneurysm neck. CFD analysis for a stented model was performed as well as for an untreated model. After the virtual intervention, a physical patch with the same design was fabricated using laser cutting techniques and micro-welded onto a commercial porous stent, creating a patient-specific asymmetric stent. This asymmetric stent was implanted into a rapid prototyped phantom of the patient-specific ACA aneurysm, which was imaged with X-ray angiography. The hemodynamics of untreated and stented aneurysms were compared both computationally and experimentally.

### Example 2

### Patient-Specific Aneurysm and Stent

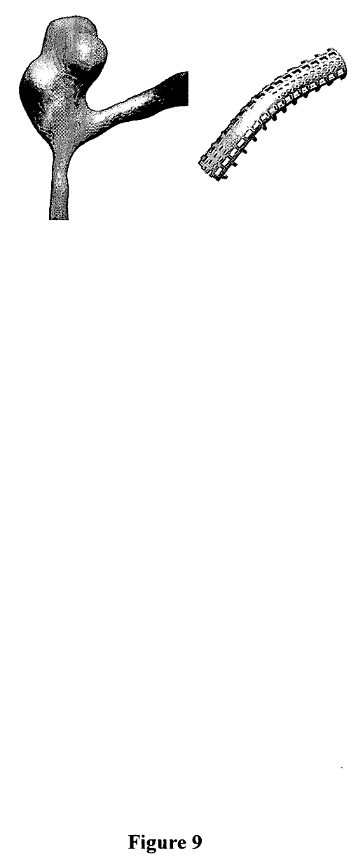

A 52-year old female patient's ACA aneurysm was selected (FIG. 9). The anatomical geometry was reconstructed from CTA images for flow analysis. Bone structures were removed from vascular anatomy. The bone-removed aneurysm geometry was segmented and smoothed for rendering. Ujiie et al. found that saccular aneurysms were more likely to rupture when the aspect ratios (AR) of the aneurysms were greater than 1.6 (Ujiie et al., “Hemodynamic Study of the Anterior Communicating Artery,” Stroke, 27:2086-2094 (1996); Ujiie et al., “Effects of Size and Shape (Aspect Ratio) on the Hemodynamics of Saccular Aneurysms: A Possible Index for Surgical Treatment of Intracranial Aneurysms,” Neurosurgery, 45(1):119-130 (1999), which are hereby incorporated by reference in their entirety). From the geometric analysis of the reconstructed aneurysm, the aspect ratio of this superior oriented ACA aneurysm was about 2.3; hence, it would be in danger of rupture. Thus, this aneurysm model was treated using an asymmetric stent patch to investigate the hemodynamic modification to reduce the postulated chance of rupture.

The patient-specific stent patch for this ACA aneurysm (FIG. 9) was designed to minimize the flow activity in the aneurysm, but on the other hand not to block the flow to peripheral vessels that might arise from the vessel walls. The local porosity of the patch was 0% (solid) at the proximal side of the aneurysm to eliminate the strong impinging flow penetration in the untreated aneurysm model. The patch porosity was also controlled to interrupt the flow that had strong momentum along the longitudinal centerline of the aneurysm neck. Away from this centerline, the patch had high porosity which allows the blood flow to the perforating arteries.

### Example 3

### CFD Simulation

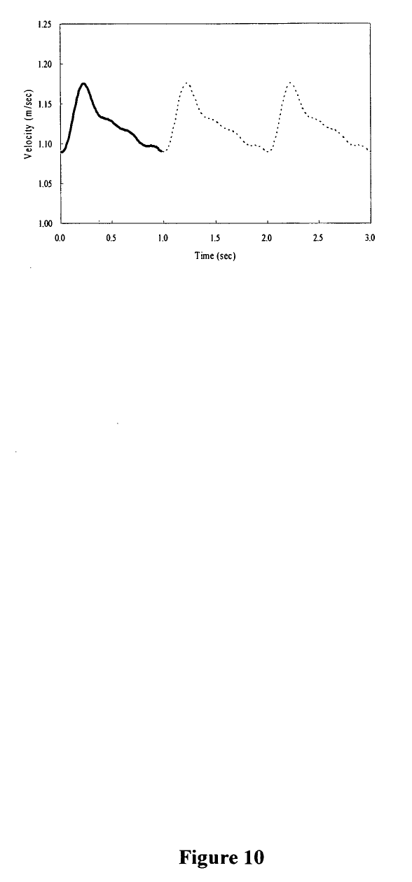

The untreated and stented aneurysm geometries were meshed with 0.6 and 1.2 million tetrahedral volume elements, respectively. The blood flow was calculated by a finite volume based CFD code, StarCD® (CD-adapco, Melville, N.Y.) under the assumption of incompressible flow. The calculation was performed with both steady and pulsatile flow conditions (FIG. 10). In addition to solving the governing equations of the flow, the scalar transport equations, which is similar to the Navier-Stokes equations but describe the motion in a scalar, were added for the virtual angiographic visualization. Therefore, sequential operations to solve the scalar transport equations were performed during each iteration. The second order accuracy was obtained by choosing a central differencing scheme for solving both flow and scalar equations. In this study, the average Reynolds number (Re) of the flow was 678, which is higher than normal but still in the range of typical flows known to occur in cerebral arteries (Burleson et al., “Computer Modeling of Intracranial Saccular and Lateral Aneurysms for the Study of Their Hemodynamics,” Neurosurgery 37(4):774-784 (1995), which is hereby incorporated by reference in its entirety). This Re is low enough to be considered laminar flow. The Womersley number of the pulsatile wave was 1.51. Blood was assumed to be Newtonian in this study because the shear rate in the artery was high, and the diameter of the artery was large (Barakat et al., “Numerical Simulation of Fluid Mechanical Distrubance Induced by Intravascular Stents,” Int. Conf. Mech. in Medi. and Bio. (2000), which is hereby incorporated by reference in its entirety). The viscosity and the density of blood in all models was 3.5 cPs and 1056 kg/m³, respectively (Aenis et al., “Modeling of Flow in a Straight Stented and Nonstented Side Wall Aneurysm Model,” J. Biomech. 199(2):206-12 (1997), which is hereby incorporated by reference in its entirety). Scalar viscosity was 6.4 cPs and density was 1320 kg/m³. The aneurysm and vessel walls were assumed to be non-compliant as was the assumption in other studies (Bando et al., “Research on Fluid-Dynamic Design Criterion of Stent Used for Treatment of Aneurysms by Means of Computational Simulation,” Comp. Fluid Dynam. J. 11(4):527-531 (2003); Cebral et al., “Efficient Simulation of Blood Flow Past Complex Endovascular Devices Using An Adaptive Embedding Technique,” IEEE Trans. Med. Imaging 24(4):468-476 (2004); Hoi et al., “Effects of Arterial Geometry on Aneurysm Growth: Three-Dimensional Computational Fluid Dynamics Study,” J. Neurosurg. 101:676-681 (2004); Shojima et al., “Magnitude and Role of Wall Shear Stress on Cerebral Aneurysm: Computational Fluid Dynamic Study of 20 Middle Cerebral Artery Aneurysm,” Stroke 35:2500-2505 (2004); Stuhne et al., “Finite-Element Modeling of the Hemodynamics of Stented Aneurysm,” J. Biomech. 126:382-387 (2004), which are hereby incorporated by reference in their entirety).

### Example 4

### Patient-Specific Phantom and Asymmetric Stent Patch

The aneurysmal flow and the patch effect on this flow were investigated using DSA images from the patient-specific phantom model. A rapid prototype phantom model was created using a stereolithography apparatus (SLA) process. The photosensitive liquid photopolymer resins were solidified by a laser to generate the patient-specific aneurysm geometry. The surface achieved for this rapid prototype phantom had 0.15 mm accuracy. Another pattern of the phantom geometry was made from wax. The wax pattern was created by a Thermojet wax printer (3D systems, Valencia, Calif.) using 0.025 mm layers. This wax pattern was submerged in liquid silicon elastomer and the elastomer was solidified. Then, a transparent elastic silicone casting was created using lost wax technique. The aneurysm in the casting was treated with an asymmetric stent.

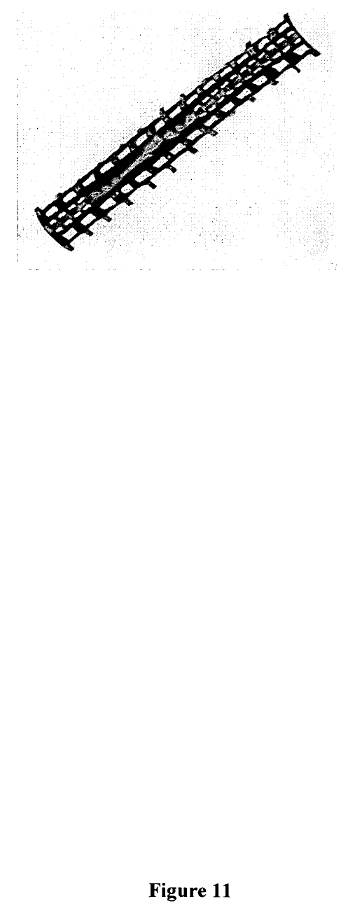

The patch geometry used for the CFD simulations was taken and a file was created which reproduced the contour with a resolution of 25 μm. This file was used in a LabView program to control the motion of a 2D motorized stage (Velmex, Bloomfield, N.Y.) and a Nd:Yag laser. The stage motion was synchronized with the laser exposure in order to cut and vaporize a pattern on a stainless steel foil with 50 μm thickness, thus creating the asymmetric patch (FIG. 11).

### Example 5

### Angiographic Flow Visualization

The rapid prototype aneurysm model was inserted in a flow loop consisting of a waveform generator, a pump, and a flow meter; the flow was activated by a heart simulating pump (Vivitro Systems Inc., Canada). A 33% glycerin-67% water mixture fluid was used to achieve dynamic similarity with the blood flow in the CFD simulation. Prior to angiographic acquisition, 3D rotational angiography of the aneurysm was performed using an Infinix angiographic C-arm (Toshiba Medical Systems Corp, Tustin, Calif.). The volume rendering was done using a Vitrea 3D station (Vital Images, Inc., Minnetonka, Minn.). The 3D rendering was used to find the orientation of the angiographic C-arm which offered the same orientation of the aneurysm as used in the CFD simulation. Further, this view was used to acquire the angiographic runs. The contrast medium was a 50% solution of water and Reno iodine contrast agent (Bracco Diagnostic, Inc, Princeton, N.J.). The flow patterns in the aneurysm were depicted by the images of contrast medium in the flow and recorded by a DSA system which has thirty frames per second frame rate. The variation of the contrast medium concentration in the aneurysm indicated the flow stasis in the aneurysm. For this, the contrast medium integration in the aneurysm sac was obtained from the DSA data. The contrast medium concentration data was normalized for quantitative comparison of flow reduction in the aneurysm between the untreated and stented case.

### Example 6

### Analysis of the Aneurysm Hemodynamics in the CFD Models

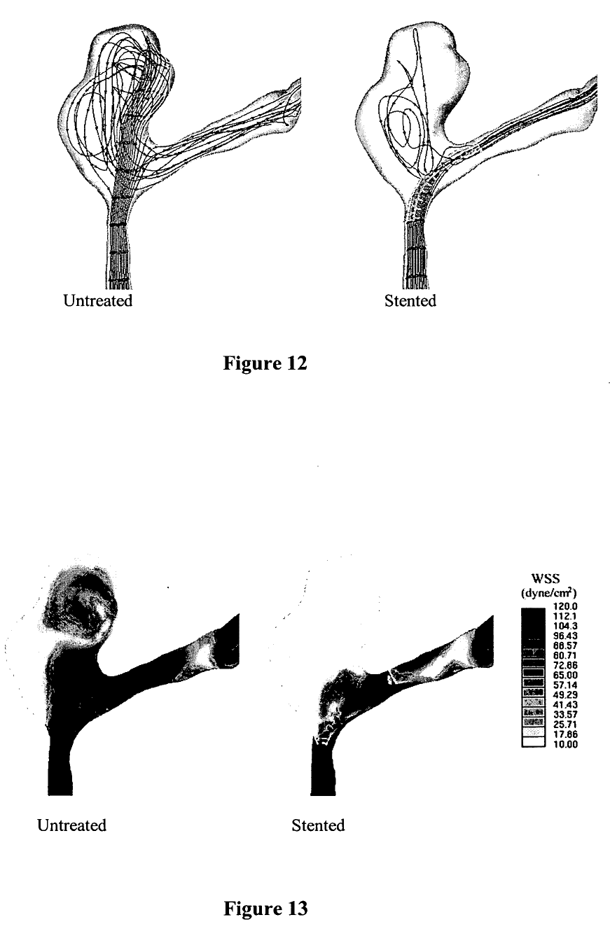

The computed aneurysmal flow patterns in the untreated and the stented models were compared. Shown in FIG. 12 are the particle paths in the steady state flow simulations. Initial points of these particles were identically selected at the inlets of both untreated and stented models. The particle paths in the untreated aneurysm showed that most of blood flow entered into the aneurysm through the proximal side at the aneurysm neck. Only a small part of the flow could bypass the aneurysm to go to the outlet in the untreated aneurysm model. Unlike the other studies (Cebral et al., “Efficient Simulation of Blood Flow Past Complex Endovascular Devices Using An Adaptive Embedding Technique,” IEEE Trans. Med. Imaging 24(4):468-476 (2004); Stuhne et al., “Finite-Element Modeling of the Hemodynamics of Stented Aneurysm,” J. Biomech. 126:382-387 (2004); Baráth et al., “Anatomically Shaped Internal Carotid Artery Aneurysm in Vitro Model for Flow Analysis to Evaluate Stent Effect,” Am. J. Neuroradiol., 25:1750-1759 (2004); Lieber et al., “Particle Image Velocimetry Assessment of Stent Design Influence on Intra-Aneurysmal Flow,” Ann. Biomed. Eng. 30:768-777 (2003), which are hereby incorporated by reference in their entirety), the role of the distal neck as a flow divider was not clear in this geometry. A major part of the untreated aneurysmal inflow impinged on and reflected off the distal wall, while a small part of the inflow directly impacted against the dome of the aneurysm. The vortex flows in the untreated aneurysm were intricate.

After the stent treatment, the blood flow pattern in the aneurysm was significantly changed. The strong inflow jet was blocked by a patch at the aneurysm neck and the direct impingement on the aneurysm wall disappeared. Most of the particle paths pass through the vessel without entering the aneurysm, and only a few of them directly penetrated the aneurysm neck. A lot of the momentum directed toward the aneurysm volume was lost during this process. Consequently, the weakened inflow led to the reduction of the intra-aneurysmal flow activity. For example, the average flow velocity magnitude in the aneurysm was reduced by 93%, and the aneurysm flow turn-over time was increased by 483% after stenting. The hemodynamic stress exerted on the aneurysm wall is substantially linked to the aneurysm growth and rupture (Kondo et al., “Cerebral Aneurysms Arising at Nobranching Sites,” Stroke 28:398-404 (1997), which is hereby incorporated by reference in its entirety). The instantaneous wall shear stress (WSS) distributions at peak systole for each aneurysm model are shown in FIG. 13. The asymmetric stent effect on aneurysm WSS is clearly demonstrated in this figure. In the untreated aneurysm, highly elevated WSS resulting from the strong impinging flow occurred at the distal wall and the dome of the aneurysm. The peak value for the untreated aneurysm WSS was 388 dyne/cm² at the distal wall. This value was about 19 times higher than normal WSS in cerebral arteries (Malek et al., “Hemodynamic Shear Stress and Its Role in Atherosclerosis,” JAMA 282(21):2035-2042 (1999), which is hereby incorporated by reference in its entirety). The asymmetric stent patch reduced the average aneurysm WSS, and the elevated WSS zone was eliminated as well. It has been found experimentally that low shear rate, which is directly related to low shear stress on the wall, promotes more thrombus formation (Hashimoto et al., “Thrombus Formation under Pulsatile Flow: Effect of Periodically Fluctuating Shear Rate,” Jpn. J. Artif. Organs 19(3):1207-1210 (1990), which is hereby incorporated by reference in its entirety). Therefore, there is a better chance of blood clotting in the stented aneurysm than the untreated aneurysm.

### Example 7

### Aneurysmal Inflow Patterns from DSA and Virtual Angiography

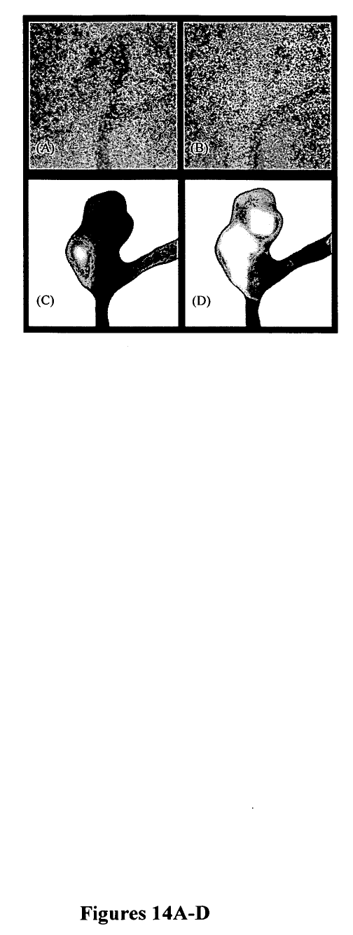

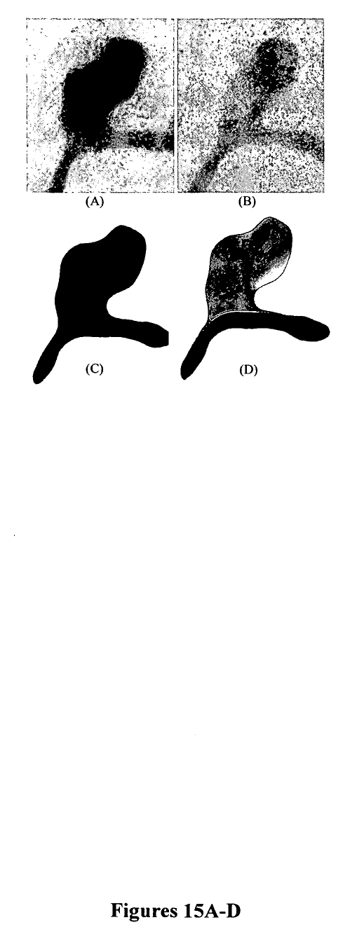

The aneurysmal inflow was visualized at the early stage of the radioopaque contrast agent injection. The contrast medium flow pattern in the aneurysm is shown in FIGS. 14A-B for the untreated and treated cases and the virtual CFD calculation results are shown in FIGS. 14C-D for the untreated and treated cases, respectively. FIGS. 14A-D are for the same time in the angiographic and calculated sequences. FIGS. 15A-D has a similar comparison for a later time in the angiographic sequences. In the comparison of the angiographical and the virtual flow visualization, the inflow patterns were consistent. The main stream of the flow entered through the proximal side at the aneurysm neck when the aneurysm was untreated. This flow met the distal wall and dispersively reflected into the deep inside of the aneurysm. The concentration of the contrast medium in the proximal region in the aneurysm was relatively lower than the other regions at this stage. Therefore, one could conclude that the flow in this region was relatively slower and the shear rate was lower than the flow in the other regions of the untreated aneurysm. The asymmetric stent patch changed the flow direction at the aneurysm neck. As a result, the direct impinging flow was eliminated and the aneurysm was hemodynamically decoupled from the artery.

### Example 8

### Flow Reduction in Untreated vs. Stented Aneurysms

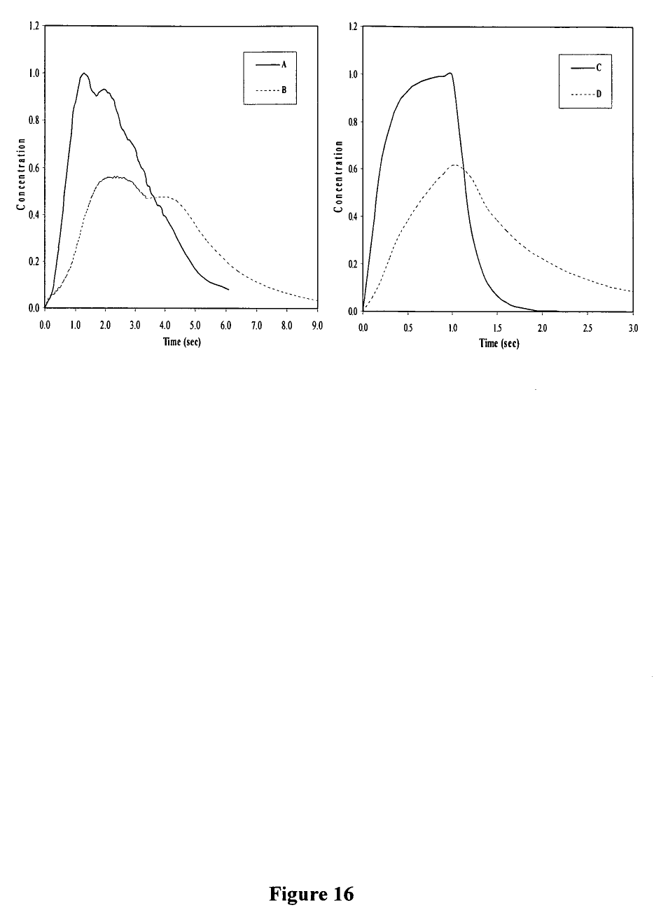

The asymmetric stent effect on the aneurysm hemodynamics was investigated experimentally using the average concentration of the contrast medium in the aneurysm as well as the flow pattern. The contrast medium concentration in the angiogram of the untreated aneurysm and the stented aneurysm were compared with those of the CFD model. FIGS. 14A-B and 15A-B are examples of the DSA images of the instantaneous contrast medium in the aneurysm, while FIGS. 14C-D and 15C-D are virtual angiographic CFD modeling results. From the image sequence, it was clear that the asymmetric stent interfered with the flow into the aneurysm. The contrast medium in the region near the aneurysm dome appeared to be somewhat trapped. The variation of the average contrast medium concentration in the aneurysm is shown in FIG. 16. In the angiographic visualization, the contrast agent was injected further upstream than in the CFD simulation and, therefore, the contrast flow duration was expanded. However, a comparison of the CFD to the angiogram shows a similar overall effect on the aneurysmal flow by the stent. By stenting, the maximum value of the average concentration of contrast medium was decreased about 44% and 38% for DSA and CFD, respectively. Conversely, the half-washout time of the contrast medium in the aneurysm was increased about 227% and 338%. From both DSA and CFD results, the aneurysmal inflow was significantly reduced and the aneurysm residence time was increased by stenting.

Aneurysm morphology is an important factor for predicting aneurysm rupture and in making a medical decision for an endovascular treatment. From the statistical analysis of ruptured and unruptured aneurysms, it has been postulated that aneurysms with large AR are more liable to rupture than those with small AR (Ujiie et al., “Effects of Size and Shape (Aspect Ratio) on the Hemodynamics of Saccular Aneurysms: A Possible Index for Surgical Treatment of Intracranial Aneurysms,” Neurosurgery, 45(1):119-130 (1999); Weir et al., “The Aspect Ratio (Dome/Neck) of Ruptured and Unruptured Aneurysms,” J Neurosurgery 99:447-451 (2003), which are hereby incorporated by reference in their entirety). Ujiie et al. found secondary flow circulation occurrence near the dome of an aneurysm which has a large aspect ratio (AR>1.6) (Ujiie et al. “Is the Aspect Ratio a Reliable Index for Prediction the Rupture of a Saccular Aneurysm?” Neurosurgery 48(3):495-503 (2001), which is hereby incorporated by reference in its entirety). According to these authors, the critically slow flow circulation in the dome of the aneurysm may cause aneurysm rupture by the following mechanism. In their discussion, the effect of enzyme digestion on the aneurysm wall remodeling was mentioned. They supposed that the low shear stress induced by the slow flow motion was correlated with atherosclerotic lesions which can degrade the integrity of the aneurysm wall and possibly cause its breakdown. An aneurysm having large AR in this study and, hence, it would be more probable to rupture. Therefore, an endovascular treatment to prevent this potential rupture was performed using a patient-specific asymmetric stent both virtually (with CFD) and experimentally with illustrated results shown in FIGS. 14A-D and 15A-D.

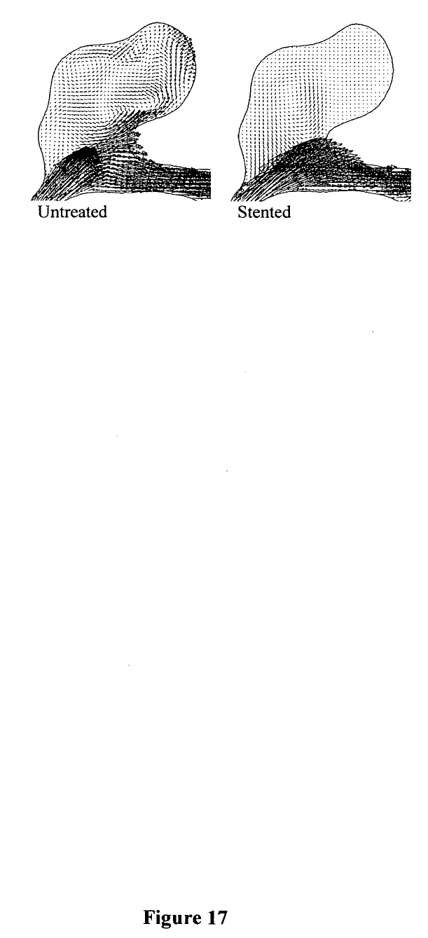

FIG. 17 illustrates the computed flow patterns in the untreated and the stented aneurysm of a patient specific case, with vectors indicating flow direction and magnitude. The flow in the untreated aneurysm was very complex and multiple vortex-like flows were found at various locations in this aneurysm. Also a strong jet-like inflow directly impinged on the confined regions of the aneurysm wall, when it was untreated. According to Cebral et al. “Characterization of Cerebral Aneurysms for Assessing Risk of Rupture By Using Patient-Specific Computational Hemodynamics Models,” Am. J. Neuroradiol., (26):2550-2559 (2005), which is hereby incorporated by reference in its entirety, the flow in ruptured aneurysms is more likely to have disturbed flow patterns, small impingement regions, and narrow jets. These aneurysmal flow characteristics were similar with the findings in the untreated aneurysm in this study.

From the CFD analysis of an idealized aneurysm on various curved vessels, Hoi et al. revealed that the aneurysm inflow and the flow impingement on the aneurysm wall increased with increasing parent vessel curvature (Hoi et al., “Effects of Arterial Geometry on Aneurysm Growth: Three-Dimensional Computational Fluid Dynamics Study,” J. Neurosurg. 101:676-681 (2004), which is hereby incorporated by reference in its entirety). From similar CFD investigations, Meng et al. found that the inflow zone was shifted from the distal to proximal side on the aneurysm neck when the parent vessel curvature increased (Meng et al., “Intravascular Stent Intervention of Cerebral Aneurysm,” BMES (2005), which is hereby incorporated by reference in its entirety). As previously shown above, in the untreated aneurysmal flow, the vessel curvature of this aneurysm was large and the impinging flow entered through the proximal neck of this aneurysm. Therefore, the asymmetric stent patch was designed to block the strong inflow at the proximal neck and possibly modify the flow to a more favorable one in this patient-specific aneurysm.

The asymmetric stent patch totally changed the hemodynamics in the aneurysm. The aneurysm flow was stabilized, and the flow pattern was simplified by the asymmetric stent placement. These simple and stable flow patterns were commonly seen in unruptured aneurysms (Cebral et al., “Characterization of Cerebral Aneurysms for Assessing Risk of Rupture By Using Patient-Specific Computational Hemodynamics Models,” Am. J. Neuroradiol., (26):2550-2559 (2005), which is hereby incorporated by reference in its entirety). Only the patch part of the asymmetric stent was modeled for CFD analysis, because the effect of the very porous part of the stent was assumed to be negligible. Since the role of the patch for aneurysm hemodynamic alteration was important, the asymmetric stent and in particular the patch must be properly placed to cover the aneurysm orifice and not to cover the terminal small perforator arteries, which could lead to local ischemia. Hence, accurate stent deployment techniques are required for the actual patient-specific stent (Ionita et al., “Microangiographic Image Guided Localization of a New Asymmetric Stent for Treatment of Cerebral Aneurysms,” SPIE 5744:354-365 (2005), which is hereby incorporated by reference in its entirety). For the purposes of the CFD study, the virtual stent patch was deformed by computer software to fit into the artery (as illustrated in FIG. 9) and was almost perfectly placed at the aneurysm neck in the CFD model.

The endovascular treatment of the patient specific aneurysms using an asymmetric stent provided desirable results in this study (Kim et al., “Evaluation of an Asymmetric Stent Patch Design for a Patient Specific Intracranial Aneurysm Using Computational Fluid Dynamic (CFD) Calculations in the Computed Tomography (CT) Derived Lumen,” Proc. of SPIE, Vol. 6143, 61432G (2006), which is hereby incorporated by reference in its entirety). Nevertheless, the biological reactions caused by the asymmetric stent can not be overlooked. It was reported that a porous stent could promote neointimal proliferation and in-stent stenosis (Krings et al., “Treatment of Experimentally Induced Aneurysms with Stents,” Neurosurgery 56:1347-1359 (2004), which is hereby incorporated by reference in its entirety). Similar reactions might occur for an asymmetric stent, but there is not enough evidence about this presently. Thus, further studies regarding the effect of asymmetric stents on the arterial wall are required.

In sum, an asymmetric stent patch was designed for a patient-specific cerebral aneurysm, and virtually implanted into the aneurysm (Kim et al., “Evaluation of an Asymmetric Stent Patch Design for a Patient Specific Intracranial Aneurysm Using Computational Fluid Dynamic (CFD) Calculations in the Computed Tomography (CT) Derived Lumen,” Proc. of SPIE, Vol. 6143, 61432G (2006), which is hereby incorporated by reference in its entirety). The asymmetric stent patch effectively blocked the strong inflow jet at the aneurysm neck and significantly reduced the flow impingement on the wall of the aneurysm. Consequently, the highly elevated WSS on the distal wall and the dome of the aneurysm was lowered down to be comparable to the normal physiological range of WSS values in cerebral artery. The aneurysmal inflow pattern computed in the CFD model qualitatively agreed with that deduced from the DSA image of the visualized flow in the phantom model. The flow stasis in the untreated and the stented aneurysm was investigated using contrast medium concentration. The variations of the contrast medium concentration derived from DSA images and virtual angiography models were analyzed. Asymmetric stent patch designs specifically for a given patient significantly reduced the maximum concentration and increased the residence time of the contrast medium in the aneurysm. It can thus be concluded that asymmetric stents are a viable intervention for treating intracranial aneurysms. Additionally, the “virtual intervention” used in this study may provide valuable clinical feedback in treatment planning as well as a better understanding of possible new treatment options when the methodology is applied retrospectively to previous clinical cases.

Although the invention has been described in detail, for the purpose of illustration, it is understood that such detail is for that purpose and variations can be made therein by those skilled in the art without departing from the spirit and scope of the invention which is defined by the following claims.

What is claimed:

1. A stent comprising:
a variable porosity, tubular structure having pores defined by structural surfaces, said tubular structure having a low porosity region in proximity to or at either end of the tubular structure, wherein the low porosity region is less porous than other regions located on the tubular structure and fully or partially obstructs passage of fluid, the low porosity region being larger than the structural surfaces between adjacent pores, wherein any arcuate path that starts at one point within the low porosity region and goes around the perimeter of the tubular structure to stop at the same point within the low porosity region must have at least a portion that is outside of the low porosity region.

2. The stent according to claim 1, wherein the low porosity region is at either end of the tubular structure.

3. The stent according to claim 1, wherein the low porosity region is in proximity to either end of the tubular structure.

4. The stent according to claim 1, wherein said end of the tubular structure has a chamfered shape.

5. The stent according to claim 4, wherein said end of the tubular structure has a shape optimal for use inside a blood vessel and/or with another stent.

6. The stent according to claim 1, wherein all cross sectional areas of said tubular structure that are perpendicular to the longitudinal axis of the tubular structure have circular shapes with identical diameters.

7. The stent according to claim 1, wherein all cross sectional areas of said tubular structure that are perpendicular to the longitudinal axis of the tubular structure have circular shapes with variable diameters.

8. The stent according to claim 7, wherein said tubular structure has a frusto-conical shape.

9. The stent according to claim 1, wherein cross sectional areas of said tubular structure that are perpendicular to the longitudinal axis of the tubular structure have variable shapes.

10. The stent according to claim 9, wherein cross sectional areas of said tubular structure that are perpendicular to the longitudinal axis of the tubular structure have elliptical or oval shapes.

11. The stent according to claim 1, wherein the low porosity region is formed by a polymer membrane patch attached to said tubular structure.

12. The stent according to claim 11, wherein the polymer membrane patch is made of polyurethane.

13. The stent according to claim 1, wherein said tubular structure comprises a cylindrical sheet with pores of variable size or shape.

14. The stent according to claim 13, wherein said tubular structure is made of a mesh material.

15. The stent according to claim 1, wherein the low porosity region has a single pore size while all other parts of the tubular structure have another larger pore size.

16. The stent according to claim 1, wherein the low porosity region has a plurality of pore sizes with the size of the pores increasing as the low porosity region transitions to other regions of the stent.

17. The stent according to claim 1, wherein said tubular structure is formed from a plurality of strut elements which are thicker, wider, and/or denser in the low porosity region.

18. The stent according to claim 17, wherein the strut elements are made of stainless steel.

19. The stent according to claim 1, wherein the low porosity region is formed by flap-like structures in the pores.

20. The stent according to claim 1, wherein the stent is balloon expandable.

21. The stent according to claim 1, wherein the stent is self-expandable.

22. The stent according to claim 21, wherein the stent is made of a superelastic or shape memory material.

23. The stent according to claim 22, wherein the superelastic or shape memory material is nitinol.

24. The stent according to claim 1, wherein the stent is marked with, or at least partially made of, a radioopaque material imageable by high resolution radiographic imaging.

25. A method of modifying blood flow within and near an opening of an aneurysm in a blood vessel comprising:
deploying one or more stents according to claim 1 near an opening of an aneurysm in a blood vessel, so that the low porosity region of the stent causes modification of blood flow within and near the opening of the aneurysm.

26. The method according to claim 25, wherein said aneurysm is located in proximity to a vessel junction wherein one or more blood vessels split or merge into one or more blood vessels.

27. The method according to claim 26, wherein said aneurysm is located in proximity to a vessel bifurcation.

28. The method according to claim 25, wherein the low porosity region of said stent is at either end of the tubular structure.

29. The method according to claim 28, wherein said deploying comprises deploying the stent so that the low porosity region at one end of the tubular structure is proximal to the opening of the aneurysm while the other end of the tubular structure is distal to the opening of the aneurysm.

30. The method according to claim 28, wherein said deploying comprises deploying the stent so that the low porosity region at one end of the tubular structure is distal to the opening of the aneurysm while the other end of the tubular structure is proximal to the opening of the aneurysm.

31. The method according to claim 25, wherein the low porosity region of said stent is in proximity to either end of the tubular structure.

32. The method according to claim 25, wherein said deploying is performed using a balloon catheter.

33. The method according to claim 25, wherein said deploying is performed by self-expansion of the stent.

34. The method according to claim 25, wherein said deploying is guided by high resolution radiographic imaging.

35. The method according to claim 25, wherein said end of the tubular structure of said stent has a chamfered shape.

36. The method according to claim 25, wherein said end of the tubular structure of said stent has a shape optimal for use inside a blood vessel and/or with another stent.

37. The method according to claim 25, wherein all cross sectional areas of said tubular structure of said stent that are perpendicular to the longitudinal axis of the tubular structure have circular shapes with identical diameters.

38. The method according to claim 25, wherein all cross sectional areas of said tubular structure of said stent that are perpendicular to the longitudinal axis of the tubular structure have circular shapes with variable diameters.

39. The method according to claim 38, wherein said tubular structure has a frusto-conical shape.

40. The method according to claim 25, wherein cross sectional areas of said tubular structure of said stent that are perpendicular to the longitudinal axis of the tubular structure have variable shapes.

41. The method according to claim 40, wherein cross sectional areas of said tubular structure of said stent that are perpendicular to the longitudinal axis of the tubular structure have elliptical or oval shapes.

42. The method according to claim 25, wherein the low porosity region of said stent is formed by a polymer membrane patch attached to said tubular structure.

43. The method according to claim 42, wherein the polymer membrane patch is made of polyurethane.

44. The method according to claim 25, wherein the tubular structure of said stent comprises a cylindrical sheet with pores of variable size or shape.

45. The method according to claim 44, wherein said tubular structure is made of a mesh material.

46. The method according to claim 25, wherein the low porosity region of said stent has a single pore size while all other parts of the tubular structure of said stent have another larger pore size.

47. The method according to claim 25, wherein the low porosity region of said stent has a plurality of pore sizes with the size of the pores increasing as the low porosity region transitions to other regions of the stent.

48. The method according to claim 25, wherein the tubular structure of said stent is formed from a plurality of strut elements which are thicker, wider, and/or denser in the low porosity region.

49. The method according to claim 48, wherein the strut elements are made of stainless steel.

50. The method according to claim 25, wherein the low porosity region of said stent is formed by flap-like structures in the pores.
